(19) United States
(12) Patent Application Publication
(10) Pub. No.: US 2020/0282001 A1
(43) Pub. Date: Sep. 10, 2020

(54) Lemon Grass Powder Extraction for Digestive Problems, Irritable Bowel Syndrome with Diarrhea, Abdominal Cramps, Abdominal Bloating, Travelers Diarrhea, Small Intestinal Bacterial Overgrowth, and Bloating

(71) Applicant: Better and Beautiful Humanity Research, LLC, Plano, TX (US)

(72) Inventor: Adnan S. Badr, Plano, TX (US)

(21) Appl. No.: 16/645,736

(22) PCT Filed: Sep. 25, 2018
(86) PCT No.: PCT/US2018/052597

§ 371 Date: Mar. 9, 2020

Related U.S. Application Data
(60) Provisional application No. 62/572,162, filed on Oct. 13, 2017.

(51) Int. Cl. A61K 36/899; A61P 1/12

(52) U.S. Cl. CPC A61K 36/899; A61P 1/12; A61K 2236/17; A61K 2236/37; A61K 2236/331

(57) ABSTRACT

The present invention includes compositions, methods of making, and methods of treating or reducing digestive problems or symptoms thereof with an improved heat activated lemon grass composition made by a method comprising combining dried lemon grass tea leaves with water; heat activating and extracting active agents in the lemon grass leaves for 35-45 minutes at 90-100° C.; separating the lemon grass leaves from the heat activated active agent in solution; vacuum or spray drying the heat activated active agents; and packaging the heat activated active agents for immediate release, wherein the heat activated active agents provide relief in less than 15 minutes from digestive problems.

### TECHNICAL FIELD

The present invention relates in general to the field of digestive problems, and more particularly, to a method for making a lemon grass powder extraction for digestive problems, irritable bowel syndrome with diarrhea, abdominal cramps, abdominal bloating, travelers diarrhea, small intestinal bacterial overgrowth, and bloating.

### BACKGROUND ART

Without limiting the scope of the invention, its background is described in connection with digestive problems.

Diarrhea annually is responsible for the mortality of almost 2.2 million people worldwide with the majority being newborns and children below the age five, around the world.

Traveler's Diarrhea (TD) and Gastroenteritis Diarrhea are usually associated with symptoms such as diarrhea, abdominal cramps and pain, nausea, vomiting, and bloating. Treatment of TD is needed to overcome abdominal cramps, abdominal discomfort, and frequent bowel movements. Therefore, conquering diarrhea is complicated and costly, especially in developing countries and third world countries. Synthetic anti-diarrhea drugs that are used for management and treatment of diarrhea such as racecadotril and loperamide have side effects such as vomiting, fever, and bronchospasm that sometimes make them impractical drugs for children below 6 years old^{[10]}. Usually, physicians increase the dose of anti-spasmodic medication to improve the efficacy of treatment which tremendously increases the side effects.

Regardless of what might be the source and etiology of the diarrhea, pathogen or non-pathogen, it may cause death if it is not managed and treated in time^{[9]}. Biological studies indicate that phytochemical constituents of lemon grass, such as flavonoids, tannins, and alkaloid have efficacy to be used as an anti-bacterial agent and antispasmodic. Moreover, affordability, fewer side effects, and easy availability of medicinal plants in nature make them good candidates for human diseases such as diarrhea^{[7]}.

Irritable Bowel Syndrome (IBS) is a functional disorder of the gastrointestinal tract with symptoms such as altered bowel routine, bloating, and cramping which is associated with chronic abdominal pain. In North America by itself IBS affects approximately 45 million adults. Irritable Bowel Syndrome can be caused by psychological problems, intestinal muscle spasms, stress, bacterial overgrowth in the small intestine, and food intolerance. As a result, sometimes altering the lifestyle and dietary habits of the diagnosed patient can be helpful in overcoming IBS symptoms.^{[1]}.

Although IBS is a common disorder in most cases, it is often not diagnosed within the first 3 to 6 months. This is due to the absence of any structural or biochemical irregularities in patient's gastrointestinal tract.^{[1]} The diagnosis usually requires multiple tests to rule out structural or physical pathology.

IBS is categorized based on its symptoms; IBS can be associated with constipation (IBS-C), diarrhea (IBS-D) or mix symptoms (IBS-M). Irritable Bowel Syndrome with Diarrhea is the most common subtype of the IBS; which by itself covers 40% of the IBS patient population.

Although Irritable bowel syndrome with diarrhea (IBS-D) is not a life-threatening disorder, it can significantly affect the patient's quality of life. IBS-D can cause a major economic burden on the government, society, and patients from the diagnostic stage to the treatment stage^{[2]}. One retrospective case study control in Health Maintenance Organization (HMO) indicates that patients with IBS have more diagnostic costs compared to the patients who are not diagnosed with IBS, and these are all due to the significant number of repetitive diagnostic tests (imaging, biological test, and procedures). IBS-D also has a negative impact on patients' work productivity and economic well-being. A patient diagnosed with IBS-D may have an anxiety of social activities due to her/his fear of not having quick access to the restroom. Also, IBS-D symptoms may cause shame for the patients in their closest relationships.

IBS-D is a static and persistent disorder, and more than 25% of the time patients have a loose, mushy, or watery stool (Based on Bristol stool Scale).

In the human body, Brain-Gut interaction and the enteric nervous system are responsible for regulating the communication between the brain and the gastrointestinal tract. As a result, any modification of signals between the brain and GI tract might cause various types of gastrointestinal symptoms such as IBS-D. Visceral hypersensitivity (VS) is defined as any abnormal pain in the inner organs due to the gas, or contraction. It is one of the main characteristics of the irritable bowel syndrome. Also altered signals in brain-gut axis affect the gastrointestinal motility in different parts of the digestive system such as the small intestine, large intestine, and rectum. Frequent bowel movements, and loose stool consistency of the IBS-D is a result of these signal alterations.

In some cases, IBS-D symptoms might be accrued by modification in the concentration of beneficial and harmful bacteria in the digestive tract. Also, increased permeability in the GI tract may be triggered by food intolerance/allergies, physical and psychological stress. Therefore, the conquering multifactorial nature of the IBS-D is complicated and costly, especially in developing countries and third world countries.

To overcome these problems, since 2004, World Health Organization (WHO) has concentrated on initiating a management program based on the herbal folks and remedies due to this knowledge that three-quarters of the world population's primary method of treatment depends on natural remedies.^{[3-4]}.

Another such treatment is found in U.S. Patent Application Publication 2013/0071474, filed by Colman, and entitled “Combinations of berberine, artemisinin, loperamide and their derivatives to treat malaria, diarrhea, travelers' diarrhea, dysentery, dengue fever, parasites, cholera and viruses.” This applicant is said to teach compositions and methods of combining berberine, artemisinin and loperamide or their derivatives in a therapeutic product for mammals suffering from malaria, diarrhea, travelers' diarrhea, dysentery, dengue fever, parasites, cholera and viruses by administration of a therapeutically effective amount of the composition.

Despite these advancements, a need remains for novel compositions and methods of making the same that can be used to immediately and effectively reduce or eliminate digestive problems.

### DISCLOSURE OF INVENTION

In one embodiment, the present invention includes a method of making an improved Cymbopogon Citratus (lemon grass) extract comprising: combining dried lemon grass tea leaves with water; heat activating and extracting active agents in the lemon grass leaves for 35-45 minutes at 90-110° C.; separating the lemon grass leaves from the heat activated active agent in solution; vacuum or spray drying the heat activated active agents; and packaging the heat activated active agents for immediate release, wherein the heat activated active agents provide relief in less than 15 minutes from digestive problems. In one aspect, the digestive problems are selected from irritable bowel syndrome with diarrhea, abdominal cramps, abdominal bloating, traveler's diarrhea, or small intestinal bacterial overgrowth (SIBO), and bloating. In another aspect, the heat activation is at 91, 92, 93, 94, 95, 96, 97, 98, or 99, 100, 101, 102, 103, 104, 105, 106, 107, 108, 109° C. In another aspect, the method further comprises the step of adding starch to the heat activated active agent is solution. In another aspect, the heat activation is for between 36, 37, 38, 39, 40, 41, 42, 43, or 44 minutes. In another aspect, the heat activation is for 40 minutes. In another aspect, the vacuum drying is in a rotary evaporator with a heating bath at 35-45° C. and a cooling coil at 3-8° C. In another aspect, the vacuum drying is in a rotary evaporator with a heating bath at 40° C. and a cooling coil at 5° C. In another aspect, the dried lemon grass tea leaves are combined with water at a weight to liquid ratio of 0.1-0.2 kilograms of dried lemon grass leaves to 4.5-5.0 liters of water. In another aspect, the dried lemon grass tea leaves are combined with water at a weight to liquid ratio of 0.16 kilograms of dried lemon grass leaves to 4.8 liters of water. In another aspect, the combination of heat activation time and the vacuum drying provides greater relief of symptoms than steeped lemon grass tea.

In another embodiment, the present invention includes a composition for treating digestive problems made by a method comprising: combining dried lemon grass tea leaves with water; heat activating and extracting active agents in the lemon grass leaves for 35-45 minutes at about 90-110° C.; separating the lemon grass leaves from the heat activated active agent is solution; vacuum or spray drying the heat activated active agents; and packaging the heat activated active agents for immediate release, wherein the heat activated active agents provide relief in less than 15 minutes from digestive problems. In one aspect, the digestive problems are selected from irritable bowel syndrome with diarrhea, abdominal cramps, abdominal bloating, traveler's diarrhea, or small intestinal bacterial overgrowth (SIBO), and bloating. In another aspect, the heat activating is at 91, 92, 93, 94, 95, 96, 97, 98, 99, 100, 101, 102, 103, 104, 105, 106, 107, 108, or 109° C. In another aspect, the method further comprises the step of adding starch to the heat activated active agent is solution. In another aspect, the heat-activating step is for between 36, 37, 38, 39, 40, 41, 42, 43, or 44 minutes. In another aspect, the heat-activating step is for 40 minutes. In another aspect, the step of vacuum drying is in a rotary evaporator with a heating bath at 35-45° C. and a cooling coil at 3-8° C. In another aspect, the step of vacuum drying is in a rotary evaporator with a heating bath at 40° C. and a cooling coil at 5° C. In another aspect, the dried lemon grass tea leaves are combined with water at a weight to liquid ratio of 0.1-0.2 kilograms of dried lemon grass leaves to 4.5-5.0 liters of water. In another aspect, the dried lemon grass tea leaves are combined with water at a weight to liquid ratio of 0.16 kilograms of dried lemon grass leaves to 4.8 liters of water. In another aspect, the combination of heat activation time and the vacuum drying provides greater relief of symptoms than steeped lemon grass tea.

In another embodiment, the present invention includes a method of treating a digestive problem comprising: identifying a subject in need of reduction of elimination of the digestive problems; and providing the subject with a heat activated and vacuum dried composition made by a method comprising: combining dried lemon grass tea leaves with water; heat activating and extracting active agents in the lemon grass leaves for 35-45 minutes at 90-110° C.; separating the lemon grass leaves from the heat activated active agent is solution; vacuum or spray drying the heat activated active agents; and packaging the heat activated active agents for immediate release, wherein the heat activated active agents provide relief in less than 15 minutes from digestive problems.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention relates to a processed medication containing Cymbopogon Citratus (lemon grass) extracted by specific hot water decoction method and converted to powder concentrate to treat and manage a multitude of digestive symptoms, in addition to Irritable Bowel Syndrome with Diarrhea (IBS-D) disorder and traveler's diarrhea with symptom such as diarrhea, bloating, cramping, spasm, and abdominal pain, and also to treat gastroenteritis with symptoms such as bloating, cramping, diarrhea and abdominal pain. The present invention can be used for controlling diarrhea caused by pathogen and non-pathogen sources in a human's body. Moreover, the present invention can be used as an alternative and complimentary anti-diarrhea, anti-spasm, anti-bloating, anti-abdominal pain, and anti-cramping. Different parts of lemon grass such as leaf, stem, and root (fresh or dry) can orally be consumed in the format of tea, essential oil and crushed leaves. The effectiveness of Cymbopogon citratus medicinal remedy is not dependent on one or two main components. Biological studies indicate that the combination of the active ingredients has a crucial role in their therapeutic effects. Current powder in pill form of lemon grass is dried leaves that were crushed or ground and stuffed in the pill. This way of introducing the lemon grass does not correlate with folk historical use of lemon grass as a tea for gastrointestinal symptoms like abdominal cramps, diarrhea and gastrointestinal upset.

The present invention eliminates steps of natural remedy preparation in a format of hot water decoction for patients in pain especially when there is no access to tea preparation tools. Also, the goal of this innovation is to introduce a reliable and easy format of a device to deliver a lemon grass remedy that is extracted by a hot water decoction method and converted to powder and/or pill form. Surprisingly, it has been found that this heat-activated and powdered extract is able to provide immediate relief to subjects, for example in just 5 to 15 minutes, with 30 minutes at the upper end. Further, the heat activated active ingredients can be provided to the subject without the need to water, which is important in places of the world where water is scarce, water is contaminated (and hence, the reason for the digestive problem(s)), or where water would be contraindicated for reducing or eliminating the digestive problem (e.g., bloating). Thus, the present invention provides two distinct advantages over the current art. First, it was recognized that the heat-activation for at least 30 minutes, but no more than 45 minutes, yielded a unique and particularly effective dosage form of the active agents in the lemon grass, which has been heretofore unrecognized, and further, that the dried heat-activated active agents provided an almost immediate relief for symptoms of digestive problems, including irritable bowel syndrome with diarrhea, abdominal cramps, abdominal bloating, travelers diarrhea and small intestinal bacterial overgrowth (SIBO), and bloating.

A dosage unit for use of the heat-activated and extracted lemon grass formulation of the present invention, may be a single compound or mixtures thereof with other compounds, e.g., another agent useful for treating digestive problems. The heat-activated and extracted lemon grass formulation of the present invention is administered in oral form using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the heat-activated and extracted lemon grass formulation of the present invention to a patient in need of therapy that includes the heat-activated and extracted lemon grass formulation.

The heat-activated and extracted lemon grass formulation is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the heat-activated and extracted lemon grass formulation may be formulated to provide, e.g., maximum and/or consistent dosing for the particular form for oral administration. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference, and the like, relevant portions incorporated herein by reference.

For example, the heat-activated and extracted lemon grass formulation may be included in a tablet or as a powder. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The heat-activated and extracted lemon grass formulation may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The heat-activated and extracted lemon grass formulation may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the heat-activated and extracted lemon grass formulation may be coupled one or more biodegradable polymers to achieve controlled release of the heat-activated and extracted lemon grass formulation, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, capsules or gelatin capsules (gelcaps) may include the heat-activated and extracted lemon grass formulation and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate and/or starch.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Suspension. An aqueous suspension is prepared for oral administration so that each 5 ml contain 50-1,000 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents.

Cymbopogon Citratus is a plant from Poaceae family with 55 species, commonly known as lemon grass, and is a perennial grass, which is mostly distributed in tropical and subtropical areas and also in the middle east. Also, they are widely distributed in Asian countries such as Indonesia, Malaysia, Africa and South American countries, due to their enormous plant diversity^{[1, 2]}. The lemon grass plant has spherical stalks with long, thin scented leaves which grow between 0.5-2 meters and the leaf height usually reaches to 100 cm long and 2 cm in width. This plant has a fibrous root with a short underground stem with a ringed segment.

The ideal environment for lemon grass to grow is tropical and subtropical (humid and warm) areas with good sunshine and a temperature range between 20-30° C. and adequate amount of rainwater (230-330 cm) per year^{[3]}.

Scientific studies indicate that Cymbopogon Citratus (lemon grass) and other family members of this plant such as Cymbopogon bombycinus, Cymbopogon ambiguus, Cymbopogon obtectus, Cymbopogon refractus, Cymbopogon citrate, Cymbopogon nardus, and Cymbopogon schoenanthus share medicinal properties due to their similar phytochemical constituents such as flavonoids, tannins, saponins, alkaloids, and other known compounds. These properties include anti-diarrhea, anti-inflammatory, anti-nociceptive, and anti-oxidants^{[3]}.

It was found that seeping and infusion of Cymbopogon Citratus (lemon grass) does not have any effect on a healthy human body, and traditionally has been used as a cuisine herb in different parts of the world^{[4]}. On the other hand, the Cymbopogon Citratus (and its family) traditionally have been reported to be useful for management and treatment of digestive disorders, bowel problems, and abdominal discomfort^{[5]}. The present invention overcomes the problems associated with current methods and preparations by providing an improved composition that is organoleptically pleasing, does not require water, or hot water, and is more effective as a result of the times and temperatures of the decoction of the lemon grass.

Furthermore, this indicates that lemon grass plant (leaf, stem, and root) in the format of decoction, infusion, and essential oil extract are desirable for treatment of irritable bowel syndrome with diarrhea (IBS-D) which has symptoms such as Diarrhea, Bloating, and Abdominal Pain. Also, Lemon grass can be used for treatment of traveler diarrhea, gastroenteritis, cramping, bloating, and abdominal paint^{[1]}.

Mechanism of action of Cymbopogon Citratus (lemon grass) extraction in any format (decoction, essential oil, infusion) in the human body is not fully recognized yet. Many studies had revealed that phytochemical and secondary metabolites constituents of lemon grass have a crucial role for their therapeutic characteristics^{[4]}. Efficacy of Cymbopogon C. (lemon grass) is dose related and it has higher inhibition in higher dosage without significant risk of toxicity for the human body. Studies point out that within the phytochemical constituents of lemon grass flavonoids, saponins, and tannin, in addition to other possible compounds, might be responsible for the anti-diarrheal and anti-spasmodic effects in the human body^{[2,5-6]}.

The present invention includes a specific method for decoction, extraction, infusion and thereafter powder concentrate of lemon grass tea that is consumed by the patients in a format of powder, gummies, capsules, tablet, roll-on, and mouth strips is necessity for treatment and provides immediate relief of symptoms. The optimal effectiveness of natural based medicinal plants is due to the combination of their active ingredients, and it is not dependent on one or two main components. As a result, this improved medicinal composition is more desired to be consumed in the whole lemon grass extract for the treatment of IBS-D, bloating, cramping, abdominal pain, dyspepsia, gastroenteritis, and traveler diarrhea^{[7]}.

Lemon Grass phytochemical tests have carried out the presence of alkaloid, tannins, saponins, flavonoids, resins, oils, steroids, glycosides, terpenoids, acidic compounds, carbohydrates, reducing sugars and proteins^{[11]}. The mechanism of action of phytochemical constituents of lemon grass is not known yet, but accumulating evidence indicates that medicinal remedies with anti-diarrhea constituents such as flavonoids, tannins, saponins, and alkaloid have the potency to inhibit the secretion in intestine, decrease intra-luminal fluid accumulation, improve the water absorption, decreases the motility of smooth muscles and in some cases delay the intestinal transit time^{[10]}. For example, tannins are natural compounds that decrease peristaltic index, which decreases the irritable bowel syndrome (IBS) symptoms^{[10]}. Flavonoids are polyphenolic compounds that are classified into 6 groups due to differences in their chemical structures. Flavonoids are highly available in nature under the names of flavonols, flavones, flavanones, catechins, anthocyanidins, and isoflavones^{[12]}. The primary skeleton of flavonoids is two benzene rings, three carbon atoms and also hydroxyl groups. The distribution and amount of these hydroxyl groups provide the variety of flavonoids in nature. Moreover, some studies have revealed that the flavonoids family has tremendous relaxing effects on contracted smooth muscles of the small and large intestine, and they have the ability to delay intestinal transit time^{[13]}. Flavonoids have antispasmodic effects that are able to manage abdominal pain and cramping caused by smooth muscle contractions in the gastrointestinal tract. Flavonoids family acts as an inhibitory agent for enzyme urease, which is a critical factor for a bacterium to survive in the acidic environment, such as the stomach^{[14,15]} Studies point out that within the phytochemical constituents of lemon grass flavonoids, saponins, and tannin may be responsible for the anti-diarrheal and anti-spasmodic effects in the human body^{[2,5,6]}.

Therapeutic preparation of Cymbopogon Citratus (Lemon Grass). All different parts of lemon grass such as leaf, root and stem (fresh or dry) can be used in folk remedies in the format of tea, decoction, steam distillation, squeeze, and diffusion. Tea format and decoction of the lemon grass is mostly used in the Middle East, West Africa, South America, and Asian countries^{[1,16]}. Nearly 80% or 85% of the world's population depends on natural based treatment due to cost and the limitation of antibacterial and other drugs^{[7,18]}. As a result, we can point out based on the traditional folk results and also new clinical studies that the lemon grass and its family may be a suitable complementary or alternative therapeutic medicine to use for treatment of IBS-D, traveler's diarrhea, small intestinal bacterial overgrowth (SIBO), acute gastroenteritis, bloating, cramping, and abdominal pain, abdominal discomfort and other digestive symptoms.

However, dry parts of lemon grass, such as the leaf and stem, have more medicinal properties than fresh parts due to the high amount of phenol, flavonoids, and other compounds. Shade drying method of therapeutic tea preparation has shown to preserve more aromatic and medicinal properties such as the antioxidant in comparison to other drying methods^{[17]}. As a result, to protect the efficacy of the lemon grass plant, we desire to use the shade dried plant leaf for the remedy of the present invention^{[3]}. Moreover, the hot water extract notably has high antioxidant properties compared to cold water extract^{[3]}.

The lemon grass decoction and altering the solution to concentrated powder format can be convenient for the individual who is suffering from the Irritable bowel syndrome with diarrhea or suffering from SIBO, traveler diarrhea and other digestive symptoms such as bloating, cramping, abdominal pain, and spasms. The powder shape of the lemon grass decoction remedy in dosage of 2 cups (16 oz) can be delivered with tablets, pills, gelcaps, capsules, gummies, thin films, pastilles, immediately disintegrating tablets, or in real urgent situations can be administered with water to shorten time to efficacy in the intestinal tract.

Another advantage of the present invention is that it cuts the steps in the preparation of a remedy (such as drying the Cymbopogon Citratus leaves), steeping in hot water, stirring time, and draining. Consequently, this makes it more convenient for patients in pain especially when there is no access to dried lemon grass leaves or no access to the tea preparation tools. Also, the secondary innovation goal indicates that lemon grass decoction powder can be delivered in the format of capsules, tablets, fast dissolving film, gelcaps, capsules, and other easy and reliable drug delivery systems for the sake of patients' comfort.

Method of Extraction for the Lemon Grass Dietary Supplement:

Batch Scale (X): To Process 0.16 Each (“X” 0.1-0.5 kg) of Lemon grass.

Theoretical yield (at 100% purity) (T)=0.021 kg (X×0.13*)

Expected yield (P)=0.017 to 0.021 kg (80% to 100%)
This ratio does not include the weight associate with the starch to the process.

| | | | 100% Wt | Assay | | | | |
|---|---|---|---|---|---|---|---|---|
| | | | Ratio | (%) | Actual Wt | | Mole | |
| NO | Material | M.W. | R | A | X × 100 × R/A | | Ratio | Spec |
| 1 | Water, purified | 18.02 | 30.0 | 100 | 4.8 | kg | | W-2 |
| 2 | Lemon grass | — | 1.0 | 100 | 0.16 | kg | | L-2 |
| 3 | Water, purified | 18.02 | N/A | 100 | Ca. 0.5 kg | | | W-2 |
| 4 | Starch, soluble | — | 0.28 | 100 | 0.045 | gr | | S-4 |
| 5 | Vegetarian | N/A | 0.63 | 100 | 100 | Ea | | C-7 |
| | Capsules | | | | | | | |

Process Instructions. Handling of all material and operational procedures are to be carried out in a well-ventilated fume hood or under appropriate ventilation.
1. 0.16 kg of dried lemon grass tea leaves is used for a production of the batch.
2. Heat 4.8 kg of charge water to 95° C. (90-110° C.). Maintain heating to maintain temperature during the steeping process.
   Start temp. 20.80
   Finish temp. 208° F. (98° C.)
3. Steep the 0.16 kg lemon grass leaves for 35-45 minutes in 95° C. water (90-110° C.) Start temp. 208.8° Finish temp. 208° F. (98° C.)
4. The solution is filtered to remove any insoluble materials. Filter the steeped lemon grass material to the 4 L filtering flask.
5. Rinse the steep tank with 0.5 kg water, and wash forward to the filtering unit.
6. Adding 0.045 gr soluble starch to the rotary evaporator flask prior to connecting to the rotary evaporator machine.
7. Apply full vacuum to the rotary evaporator with heating bath at 40° C. and cooling coil at 5° C. which this process helps to dryness of the remedy. Optionally, the heat-activated extract is spray dried.
8. Remove the solids from the round bottom flask of the Rotary Evaporation machine and grind it with a mortar and pestle.
9. Load 100# of vegetarian capsules with the powder prepared by process number 8 and fill and compact it until the capsule is totally full. Seal the capsules.
10. Inspect each capsule for uniformly and appropriate fill level.
11. Charge capsule to bottle, seal and label.

Case Study 1: 50-year-old female has recurrent diarrhea and urgency. She had also abdominal cramps, with a history of (H/O) irritable bowel syndrome (IBS) with diarrhea. Used Lemon grass pill made according to the present invention was used twice a day for 3 days. Her diarrhea improved significantly. Stopped the medicine and her diarrhea, pain and cramps started again.

Case Study 2: 54-year-old female has H/O alternating diarrhea with constipation and IBS. She had abdominal cramps and bloating with that took lemon grass extract made according to the present invention twice daily for 5 days. Her bowel habits significantly improved. She stopped the lemon grass extract and all the symptoms came back.

Case Study 3: 35-year-old female had food poisoning and acute infectious gastroenteritis diarrhea. Took lemon grass extract pill made according to the present invention twice a day for 2 days. Symptoms improved within 3 hours after taking the first pill. Symptoms resolved completely after the third dose.

Case Study 4: 51-year-old male had abdominal pain and gas and bloating and diarrhea after eating cafeteria food. Took one pill of lemon grass extract made according to the present invention and symptoms were resolved in 6 hours.

Case Study 5: 33-year-old female had gastroenteritis with diarrhea and abdominal pain and cramping. Took lemon grass extract made according to the present invention twice a day for 2 days. Symptoms improved by 90% by the second day.

Case Study 6: 27-year-old male had H/O IBS with diarrhea and bloating, abdominal pain, urgency and cramping. Took lemon grass extract made according to the present invention twice a day for 14 days. Symptoms improved tremendously and nearly resolved. Stopped lemon grass extract and symptoms came back.

Case Study 7: 51-year-old male had symptoms of bloating, diarrhea and upset abdominal pain after eating restaurant food. Symptoms were getting worse progressively. Symptoms did not get better and persisted×3 days. Took lemon grass extract made according to the present invention twice a day for 2 days and symptoms resolved completely in 48 hours.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Olorunnisola, S. K, H. T Asiyanbi, A. M Hammed, and S. Simsek. “Biological Properties of Lemon grass: An Overview.” International Food Research Journal 21(2): 455-462 (2014).
2. Osheke Shekins Okere, Janet Olayemi Sangodele, Oluwatosin Grace Tade, Olabisi T. Obafemi, and John Adeolu Falode. “Anti-diarrhea Potential and Acute Toxicity Studies of Methanolic Extract of Vemonia Amygdalina and Cymbopogon Citratus against Castor Oil Induced Diarrhea Model in Rats.” International Journal of Biochemistry Research & Review 6(2): 46-52, 2015, Article No. IJBcRR.2015.036 ISSN: 2231-086X.
3. Vanisha S. Nambiar, and Hema Matela. “Potential Functions of Lemon Grass (Cymbopogon Citratus) in Health and Disease.” International Journal of Pharmaceutical & Biological Archives 2012; 3(5): 1035-1043.
4. Gustavo Costa, Fatima Nunes, Carla Vitorino, Joao Jose Sousa, Isabel Vitória FigueiredoVitória, and Maria Teresa Batista. “Validation of a RP-HPLC Method for Quantitation of Phenolic Compounds in Three Different Extracts from Cymbopogon Citratus.” Research Journal of Medicinal Plant 9 (7): 331-339, 2015 ISSN 1819-3455/DOI: 10.3923/rjmp.2015.331.339 © 2015 Academic Journals In.
5. K. Abubakar, M. R. Abubakar, J. C. Ugwah-Oguejiofor, A. A. Muhammad, M. Usman, and H. E. Mshelia. “Antidiarrhoel Activity of the Saponin and Flavonoid Fractions of Anarcadium Occidentale Leaves in Albino Rats.” Advancement in Medicinal Plant Research Vol. 3(1), Pp. 23-28, February 2015 ISSN: 2354-2152 Full Length Research Paper.
6. H. Hanisa, H. Hadijah, A. Rasedee, and A. S. Tarmizi. “Sub-acute Oral Administration of Cymbopogon Citratus Stem Infusion and Its Effects on Blood Biochemical Parameters, Body and Organ Weights in Rats.” J. Trop. Agric. and Fd. Sc. 39(1)(2011).
7. Abhijit Balasaheb Shinde, and Yogini Ramkrishna Mulay. “Phytochemical Analysis and Antibacterial Properties of Some Selected Indian Medicinal Plants.” International Journal of Current Microbiology and Applied Sciences, ISSN: 2319-7706 Volume 4 Number 3 (2015) Pp. 228-235.
8. Brian E. Lacy, Kirsten Weiser, and Ryan De Lee. “The Treatment of Irritable Bowel Syndrome.” Therapeutic Advances in Gastroenterology, (2009) 2(4) 221-238 DOI: 10.1177/1756283X09104794.
9. “Review Research on The Literature of Diarrhea Disease in China (1990-2004).” National Center for Rural Water Supply Technical Guidance, China CDC December, 2005.
10. Wondmagegn Tamiru Tadesse, Abebe Ejigu Hailu, Abyot Endale Gurmu, and Abraham Fikru Mechesso. “Experimental Assessment of Antidiarrheal and Antisecretory Activity of 80% Methanolic Leaf Extract of Zehneria Scabra in Mice.” Tadesse Et Al. BMC Complementary and Alternative Medicine 2014, 14:460.
11. SA Chime, S A Brown, C E C Ugwoke, C O Agubata, J O Ubah, and G C Onunkwo. “Drug Invention Today ISSN: 0975-7619.” Formulation of Methanolic Extract of Cymbopogon Citratus Tablets: In Vitro Evaluation.
12. Marcelly Barbosa Da Rocha, Fibia Valeria Menezes Souza, Charlez Dos Santos Estevam, Cosimo Pizza, Antônio Euzébio Goulart Sant'ana, and Rosilene Moretti Marcal. “Antispasmodic Effect of 4′-methylepigallocatechin on Guinea Pig Ileum.” 2012 Elsevier B. V., Fitoterapia 83 (2012) 1286-1290.
13. Smain Amira, Alessandra Rotondo, and Flavia Mule. “Relaxant Effects of Flavonoids on the Mouse Isolated Stomach: Structure-activity Relationships.” European Journal of Pharmacology 599 (2008) 126-130.
14. Bruna Vidal Bonificio, Matheus Aparecido Dos Santos Ramos, Patricia Bento Da Silva, and Tais Maria Bauab. “Antimicrobial Activity of Natural Products against Helicobacter Pylori: A Review.” Bonificio Et Al. Annals of Clinical Microbiology and Antimicrobials 2014, 13:54.
15. Iwona Konieczna, Paulina, Marek Kwinkowski, Beata Kolesiska, Justyna Fr, Zbigniew Kami, and Wies. “Bacterial Urease and Its Role in Long-Lasting Human Diseases.” Current Protein and Peptide Science, 2012, 13, 789-806.
16. Partiban Subramanian, Che Wan Imanina Che Wan Takwa, and Emelia Ahmad Zubair. “Chemical Composition and Antibacterial Activity of Essential Oil of Cymbopogon Citratus and Cymbopogon Nardus against Enterococcus Faecalis.” International Journal of Biosciences IJB 1, Vol. 6, No. 9, P. 9-17, 2015.
17. Fhatuwani N. Mudau, and Wonder Ngezimana. “Effect of Different Drying Methods on Chemical Composition and Antimicrobial Activity of Bush Tea (Athrixia Phylicoides).” INTERNATIONAL JOURNAL OF AGRICULTURE & BIOLOGY ISSN Print: 1560-8530; ISSN Online: 1814-9596 13-750/2014/16-5-1011-1014.
18. Abhijit Balasaheb Shinde, and Yogini Ramkrishna Mulay. “Phytochemical Analysis and Antibacterial Properties of Some Selected Indian Medicinal Plants.” International Journal of Current Microbiology and Applied Sciences, ISSN: 2319-7706 Volume 4 Number 3 (2015) Pp. 228-235.
19. Vareishang Tangpu, and Arun K. Yadav. “ANTIDIARRHOEAL ACTIVITY OF CYMBOPOGON CITRATUS AND ITS MAIN CONSTITUENT, CITRAL.” Pharmacologyonline 2: 290-298 (2006).

What is claimed is:

1. A method of making an improved Cymbopogon Citratus (lemon grass) extract comprising:
combining dried lemon grass tea leaves with water;
heat activating and extracting active agents in the lemon grass leaves for 35-45 minutes at 90-110° C.;
separating the lemon grass leaves from the heat activated active agent is solution;
vacuum or spray drying the heat activated active agents; and
packaging the heat activated active agents for immediate release, wherein the heat activated active agents provide relief in less than 15 minutes from digestive problems.

2. The method of claim 1, wherein the digestive problems are selected from irritable bowel syndrome with diarrhea, abdominal cramps, abdominal bloating, traveler's diarrhea, small intestinal bacterial overgrowth, or bloating.

3. The method of claim 1, wherein the heat activation is at 91, 92, 93, 94, 95, 96, 97, 98, or 99, 100, 101, 102, 103, 104, 105, 106, 107, 108, 109° C.

4. The method of claim 1, further comprising the step of adding starch to the heat activated active agent is solution.

5. The method of claim 1, wherein the heat activation is for between 36, 37, 38, 39, 40, 41, 42, 43, or 44 minutes.

6. The method of claim 5, wherein the heat activation is for 40 minutes.

7. The method of claim 1, wherein vacuum drying is in a rotary evaporator with a heating bath at 35-45° C. and a cooling coil at 3-8° C.

8. The method of claim 7, wherein vacuum drying is in a rotary evaporator with a heating bath at 40° C. and a cooling coil at 5° C.

9. The method of claim 1, wherein the dried lemon grass tea leaves are combined with water at a weight to liquid ratio of 0.1-0.2 kilograms of dried lemon grass leaves to 4.5-5.0 liters of water.

10. The method of claim 9, wherein the dried lemon grass tea leaves are combined with water at a weight to liquid ratio of 0.16 kilograms of dried lemon grass leaves to 4.8 liters of water.

11. The method of claim 1, wherein the combination of heat activation time and the vacuum drying provides greater relief of symptoms than steeped lemon grass tea.

12. A composition for treating digestive problems made by a method comprising:
combining dried lemon grass tea leaves with water;
heat activating and extracting active agents in the lemon grass leaves for 35-45 minutes at about 90-110° C.;
separating the lemon grass leaves from the heat activated active agent is solution;
vacuum or spray drying the heat activated active agents; and
packaging the heat activated active agents for immediate release, wherein the heat activated active agents provide relief in less than 15 minutes from digestive problems.

13. The method of claim 12, wherein the digestive problems are selected from irritable bowel syndrome with diarrhea, abdominal cramps, abdominal bloating, traveler's diarrhea, small intestinal bacterial overgrowth, or bloating.

14. The method of claim 12, wherein the heat activating is at 91, 92, 93, 94, 95, 96, 97, 98, 99, 100, 101, 102, 103, 104, 105, 106, 107, 108, or 109° C.

15. The method of claim 12, further comprising the step of adding starch to the heat activated active agent is solution.

16. The method of claim 12, wherein the step of heat activating is for between 36, 37, 38, 39, 40, 41, 42, 43, or 44 minutes.

17. The method of claim 16, wherein the step of heat activating is for 40 minutes.

18. The method of claim 12, wherein the step of vacuum drying is in a rotary evaporator with a heating bath at 35-45° C. and a cooling coil at 3-8° C.

19. The method of claim 18, wherein the step of vacuum drying is in a rotary evaporator with a heating bath at 40° C. and a cooling coil at 5° C.

20. The method of claim 12, wherein the dried lemon grass tea leaves are combined with water at a weight to liquid ratio of 0.1-0.2 kilograms of dried lemon grass leaves to 4.5-5.0 liters of water.

21. The method of claim 20, wherein the dried lemon grass tea leaves are combined with water at a weight to liquid ratio of 0.16 kilograms of dried lemon grass leaves to 4.8 liters of water.

22. The method of claim 12, wherein the combination of heat activation time and the vacuum drying provides greater relief of symptoms than steeped lemon grass tea.

23. A method of treating a digestive problem comprising:
identifying a subject in need of reduction of elimination of the digestive problems; and
providing the subject with a heat activated and vacuum dried composition made by a method comprising:
combining dried lemon grass tea leaves with water;
heat activating and extracting active agents in the lemon grass leaves for 35-45 minutes at 90-110° C.;
separating the lemon grass leaves from the heat activated active agent is solution;
vacuum or spray drying the heat activated active agents; and
packaging the heat activated active agents for immediate release, wherein the heat activated active agents provide relief in less than 15 minutes from digestive problems.
